(19) United States
(12) Patent Application Publication
(10) Pub. No.: US 2008/0119927 A1
(43) Pub. Date: May 22, 2008

(54) Stent Coating Including Therapeutic Biodegradable Glass, and Method of Making

(75) Inventor: Joseph Lessar, Coon Rapids, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/561,117

(22) Filed: Nov. 17, 2006

(51) Int. Cl. A61F 2/82; A61L 33/00

(52) U.S. Cl. 623/1.42; 427/2.25

(57) ABSTRACT

A biocompatible polymeric coating composition for a stent having biodegradable glass spheres housing a therapeutic agent. The biodegradable glass spheres provide controlled, sustained release of the therapeutic agent in vivo. The biocompatible polymeric coating may be prepared without the use of a co-solvent.

### FIELD OF THE INVENTION

The invention relates generally to the field of implantable medical devices. More particularly, the invention relates to an intraluminal stent including a polymeric coating having a therapeutic agent contained within biodegradable glass spheres.

### BACKGROUND OF THE INVENTION

Prosthetic devices, such as stents or grafts, may be implanted during interventional procedures such as balloon angioplasty to reduce the incidence of vessel restenosis. To improve device effectiveness, stents may be coated with one or more therapeutic agents providing a mode of localized drug delivery. The therapeutic agents are typically intended to limit or prevent restenosis. For example, anti-thrombogenic agents such as heparin or clotting cascade IIb/IIIa inhibitors (e.g., abciximab and eptifibatide) may be coated on the stent, thereby diminishing thrombus formation. Such agents may effectively limit clot formation at or near the implanted device. Some anti-thrombogenic agents, however, may not be effective against intimal hyperplasia. Therefore, the stent may also be coated with anti-proliferative agents or other compounds to reduce excessive endothelial re-growth. Therapeutic agents provided as coating layers on implantable medical devices may effectively limit restenosis and reduce the need for repeated treatments. Therapeutic agents that provide other benefits, such as anti-plaque agents, e.g., naproxen and ibuprofen, also be may desirably coated onto a stent.

Several strategies have been developed for coating one or more therapeutic agents onto the stent surface. Standard methods may include dip coating, spray coating, and chemical bonding. The therapeutic agent coating may be applied as a mixture, solution, or suspension of polymeric material and/or drugs dispersed in an organic vehicle or a solution or partial solution. However, the creation of a stent coating such that a drug may be delivered in a reliable but controlled manner presents many challenges, particularly the need to dissolve the drug inside the polymer carrier. Such drug dissolution often requires the use of solvents to dissolve the drug, and further solvents or co-solvents to dissolve the polymer. As such, finding the right solvents with the right polymer to deliver the right drug can be difficult to achieve. What is needed is a drug-eluting polymeric coating for a stent that does not require the use of co-solvents between the drug and the polymer carrier.

Water-soluble, viz., biodegradable, glasses have been utilized for a variety of medical, cosmetic and other purposes. For example, UK Patent Specifications Nos. 1,565,906, 2,079,152, 2,077,585 and 2,146,531, describe the dissolution of glasses impregnated with various agents such as drugs, hormones, insecticides, spermicides, and fungicides to provide controlled release of these agents. The glass can be in the form of an implant or bolus. WO 98/44965, describes a water-soluble biodegradable glass composition containing various active agents, e.g., antimicrobials such as antibiotics and metal compounds, e.g., silver oxide, silver orthophosphate, steroids, painkillers, etc., which is used for implantation in soft tissue. U.S. Pat. No. 6,881,766 describes sutures and polymeric coatings for sutures made from therapeutic absorbable glass containing silver to promote wound repair.

The aforementioned references describe the use of water-soluble glass for certain implant applications, sutures, wound dressings, and treating infections. However, there is no indication in the references of a polymeric coating composition for a stent having a therapeutic agent in biodegradable glass for providing controlled, sustained release of the therapeutic agent, wherein the coating can be more simply prepared without the use of a co-solvent.

### BRIEF SUMMARY OF THE INVENTION

An embodiment of the present invention is a drug-eluting stent having a coating that includes a biocompatible polymer with biodegradable glass spheres containing a therapeutic material dispersed therein. The biodegradable glass spheres may be formed from an alkali or alkaline earth metal oxide, wherein in various embodiments, the alkali or alkaline earth metal oxide may be one of sodium oxide, potassium oxide, calcium oxide, magnesium oxide, and combinations thereof. The therapeutic material may be, for example, one of an anti-proliferative agent, anti-clotting agent, anti-plaque agent and combinations thereof. In an embodiment, the biocompatible polymer is a bioabsorbable polymer, which may be one of poly(L-lactide), poly(D,L-lactide), polycaprolactone, polyoretheresters and nylon with metallic particles dispersed therein. In another embodiment, the biocompatible polymer is a biostable polymer, which may be one silicone, polyurethane, polyethylene, and polysulfone.

A method of making a drug-eluting stent according to the present invention includes providing a stent for implantation in a body lumen and applying to the stent a coating composition consisting essentially of biodegradable glass spheres containing a therapeutic agent and a biocompatible polymer. In embodiments of the present invention, the biodegradable glass spheres may be one of least one alkali or alkaline earth metal oxide, such as, sodium oxide, potassium oxide, calcium oxide, magnesium oxide, and combinations thereof. The therapeutic agent may be, for example, one of an anti-proliferative agent, anti-clotting agent, anti-plaque agent and combinations thereof. The method of making the drug-eluting stent further includes using a bioabsorbable polymer, such as, poly(L-lactide), poly(D,L-lactide), polycaprolactone, polyoretheresters and nylon as the biocompatible polymer. In another embodiment, the method includes using a biostable polymer, such as, silicone, polyurethane, polyethylene, or polysulfone as the biocompatible polymer.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a perspective view of an exemplary stent in accordance with an embodiment of the present invention.

FIG. 2 is a schematic cross-sectional view of a stent strut of the stent of FIG. 1 showing a coating in accordance with an embodiment of the present invention.

### DETAILED DESCRIPTION OF THE INVENTION

Described herein are coating compositions including biodegradable glass which are adapted for coating stents, and stents coated with such compositions. The incorporation of biodegradable glass containing a therapeutic agent (also known herein as therapeutic biodegradable glass) in association with stent coatings herein provides a unique sustained release dosage form for delivery within a body lumen. A coating composition for a stent is provided that is prepared from a biocompatible, biostable or bioabsorbable polymer and therapeutic biodegradable glass, wherein the coating composition is adapted to coat the stent. A method for preparing the stent coating is provided that involves dispersing biodegradable glass in a biocompatible, bioabsorbable or biostable polymer without the use of a co-solvent.

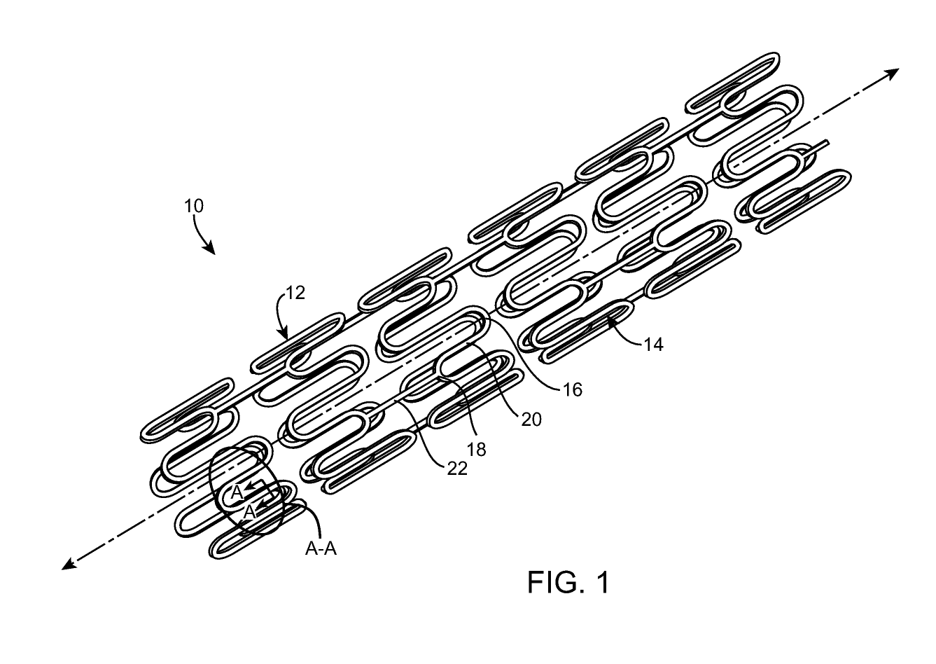

FIG. 1 illustrates an exemplary stent 10 in accordance with an embodiment of the present invention. Stent 10 is a patterned tubular device that includes a plurality of radially expandable cylindrical rings 12. Cylindrical rings 12 are formed from struts 14 formed in a generally sinusoidal pattern including peaks 16, valleys 18, and generally straight segments 20 connecting peaks 16 and valleys 18. Connecting links 22 connect adjacent cylindrical rings 12 together. In FIG. 1, connecting links 22 are shown as generally straight links connecting a peak 16 of one ring 12 to a valley 18 of an adjacent ring 12. However, connecting links 22 may connect a peak 16 of one ring 12 to a peak 16 of an adjacent ring, or a valley to a valley, or a straight segment to a straight segment. Further, connecting links 22 may be curved. Connecting links 22 may also be excluded, with a peak 16 of one ring 12 being directly attached to a valley 18 of an adjacent ring 12, such as by welding, soldering, or the manner in which stent 10 is formed, such as by etching the pattern from a flat sheet or a tube. It will be appreciated by those ordinary skill in the art that stent 10 of FIG. 1 is merely an exemplary stent and that stents of various forms and methods of fabrication can be used in accordance with various embodiments of the present invention. For example, in a typical method of making a stent, a thin-walled, small diameter metallic tube is cut to produce the desired stent pattern, using methods such as laser cutting or chemical etching. The cut stent may then be de-scaled, polished, cleaned and rinsed. Some examples of methods of forming stents and structures for stents are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,935,162 to Dang, U.S. Pat. No. 6,090,127 to Globerman, and U.S. Pat. No. 6,730,116 to Wolinsky et al., each of which is incorporated by reference herein in its entirety.

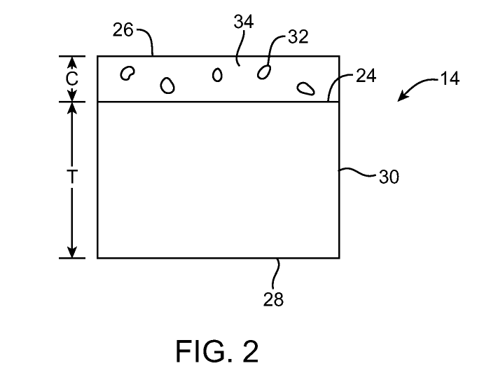

FIG. 2 is a schematic of a cross-sectional view taken at A-A of FIG. 1 that depicts stent strut 14 of stent 10 having a coating 26 in accordance with an embodiment of the present invention. Strut 14 has a suitable thickness T between the stent outer surface 24 and an inner surface 28. Typically, thickness T may be in the range of approximately 50 μm (0.002 inches) to 200 μm (0.008 inches). In various embodiments of the present invention, a cross-sectional view of connecting links 22 may be similar to strut 14, or may be different. For example, a thickness of connecting links 22 may be different than strut 14 of cylindrical rings 12 for variable flexibility between the rings 12 and connecting links 22. A specific choice of thickness for struts 14 and links 22 depends on several factors, including, but not limited to, the anatomy and size of the target lumen.

Coating 26 has a coating thickness C, wherein coating thickness C may be in the range of approximately <1 μm (0.00004 inches) to 25 μm (0.001 inches), for example. Coating 26 includes a plurality of biodegradable glass spheres 32, which include a therapeutic material dispersed there through or contained therein, and a biocompatible polymer 34. In the embodiment of FIG. 2 only outer surface 24 is shown coated by coating 26. However it should be understood that in various other embodiments, all or portions of outer surface 24, inner surface 28, and/or side surfaces 30 may be coated with coating 26, as may be desired to achieve various dosages of the therapeutic agent.

Typical materials used for stent 10 are metals or alloys, examples of which include, but are not limited to, stainless steel, “MP35N,” “MP20N,” nickel titanium alloys such as nitinol (e.g., ELASTINITE® by Advanced Cardiovascular Systems, Inc., Santa Clara, Calif.), tantalum, platinum-iridium alloy, gold, magnesium, or combinations thereof. “MP35N” and “MP20N” are trade names for alloys of cobalt, nickel, chromium and molybdenum available from standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum.

Biodegradable glass is incorporated in all aspects and embodiments herein. While glass, in general, is a durable material, the structure of glass can be made soluble in water and body fluids mainly by the addition of glass modifiers. The rate of dissolution of the biodegradable glass in water and body fluids can be arbitrarily controlled as described below. Thus, incorporation of therapeutic agents into biodegradable glass (therapeutic biodegradable glass) provides a vehicle for gradual release of desired therapeutic agents from the glass as the glass dissolves. Accordingly, stents coated with compositions including a biodegradable glass can provide controlled, sustained release of a therapeutic agent over a selected period of time.

Water-soluble glasses are well-known in the art and are described, e.g., in U.S. Pat. Nos. 5,330,770, 5,290,544, and 5,470,585, each being incorporated herein by reference. Typically, water-soluble or biodegradable glasses are made of one or two glass-forming oxides also known as glass formers, e.g., silicon dioxide, boric oxide, and phosphorus pentoxide in combination with one or more of glass modifiers, such as calcium oxide, sodium oxide, potassium oxide, zinc oxide, barium oxide, magnesium oxide, and mixtures thereof. Water-soluble glasses are also commercially available by, for example, Giltech Ltd of Scotland. Biodegradable glasses utilized in accordance with this disclosure are biocompatible, which means that the glasses do not elicit substantially adverse affects, e.g., undue toxicity or undue irritation, when implanted into living tissue.

The composition of the biodegradable glass can be specifically formulated to achieve a particular dissolution rate. The rate of dissolution is controlled by the ratio of glass modifier to glass former and by the relative amount of the glass modifiers in the glass. Generally, the glass dissolution rate decreases as the concentration of modifier increases. The biodegradable glasses employed in the invention may be those based upon P₂O₅ as the network former, and which contain at least one alkali or alkaline earth metal oxide such as sodium oxide, potassium oxide, calcium oxide, magnesium oxide, and the like. Accordingly, the solubility rate (in aqueous media) is increased by increasing the proportion of alkali metal oxides (i.e., Na₂O and K₂O), and is decreased by increasing the proportion of alkaline earth metal oxides (CaO and MgO). As such, within certain limits, the solubility rate of the glass can be varied. Other oxides can be added, in small amounts, if desired. For example, small amounts of SiO₂, B₂O₃, ZnO can be added for the purpose of retarding the dissolution rate for certain applications, or for enhancing processability.

As mentioned above, a therapeutically effective amount of a therapeutic agent may be incorporated into the biodegradable glass, which is delivered at a desired site upon dissolution of the glass. Therapeutic agent refers to one or more beneficial substances, e.g., those which aid the natural healing process and/or prevent restenosis. In accordance with one embodiment of the present invention, a therapeutic agent herein is incorporated into biodegradable glass spheres during or after manufacture of the glass spheres. Accordingly, one skilled in the art will appreciate that useful therapeutic agents herein should not be adversely affected by the glass-manufacturing process, i.e., they will remain biologically active. Suitable therapeutic agents include, but are not limited to, anti-proliferative agents, e.g., repromicin, anti-clotting agents, e.g., plasminogen activators, and/or anti-plaque agents, e.g., naproxen and ibuprofen.

The amount of therapeutic agent utilized in the biodegradable glass will depend on the conditions of use and the desired rate of release from the glass. A therapeutically effective amount of a therapeutic agent is the amount necessary to achieve desired minimal therapeutic activity. The higher the concentration of therapeutic agent contained in the glass, the higher the amount of the agent's release. In addition, by controlling the speed of glass dissolution, more or less therapeutic activity may be achieved. Faster dissolution results in more rapid release of the therapeutic agent. As used herein, therapeutic biodegradable glass refers to biodegradable glass, as defined herein, having a therapeutically effective amount of a therapeutic agent.

Bioabsorbable polymers and biostable polymers are utilized in accordance with various embodiments of the present invention. As used herein, “bioabsorbable polymer” refers to a polymer or copolymer which is absorbed by the body. “Biostable polymer” refers to a polymer or copolymer which remains in the body without substantial bio-erosion. Both bioabsorbable polymers and biostable polymers for use herein should be biocompatible. Suitable bioabsorbable polymers include, but are not limited to, poly(L-lactide), poly(D,L-lactide), polycaprolactone, polyoretheresters and nylons, if metal particles are present as a catalyst. Suitable biostable polymers include, but are not limited to, silicones, polyurethanes, polyethylenes, and polysulfones.

The coating compositions for stents can be prepared by dispersing therapeutic biodegradable glass in the biocompatible, bioabsorbable polymer or biocompatible, biostable polymer described above using any conventional technique known to one skilled in the art. In one embodiment, the therapeutic biodegradable glass in capsule or sphere form can be combined with the bioabsorbable or biostable polymer and thoroughly mixed using a homogenizer. In another embodiment, the therapeutic biodegradable glass and bioabsorbable polymer can be mixed together in powder or pellet form and then suspended using a solvent or suspending agent suitable for suspending the polymer. Because the glass capsule shields the therapeutic material from the polymer, the need for use of a co-solvent between the therapeutic material and polymer is eliminated, which simplifies preparation of the polymeric coating and allows for a greater number of therapeutic materials to be used with any suitable polymer. Prior to and/or during its application onto the stent, the coating composition can be agitated to ensure that the therapeutic biodegradable glass is uniformly distributed throughout the composition. The coating composition can be applied to the stent in any number of ways. Suitable techniques for applying the coating composition to the stent include, but are not limited to dipping, spraying, wiping and brushing. The amount of coating composition applied to the stent will vary depending on the structure, size and composition of the stent.

In addition to the stent incorporated by reference above, the aforementioned stent coatings may be applied to any of the stents disclosed in U.S. Pat. No. 5,133,732, U.S. Pat. No. 5,776,161, U.S. Pat. No. 6,113,627, and U.S. Pat. No. 6,663,661, which are incorporated by reference herein in their entirety.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A drug-eluting stent having a coating comprising:
biodegradable glass spheres containing a therapeutic material; and
a biocompatible polymer.

2. The stent coating of claim 1, wherein the biocompatible polymer is a bioabsorbable polymer selected from the group consisting of poly(L-lactide), poly(D,L-lactide), polycaprolactone, polyoretheresters and nylon with metallic particles dispersed therein.

3. The stent coating of claim 2, wherein the biodegradable glass spheres are comprised of at least one alkali or alkaline earth metal oxide.

4. The stent coating of claim 3, wherein the alkali or alkaline earth metal oxide is selected from the group consisting of sodium oxide, potassium oxide, calcium oxide, magnesium oxide, and combinations thereof.

4. The stent coating of claim 4, wherein the therapeutic material is selected from the group consisting of anti-proliferative agents, anti-clotting agents, anti-plaque agents and combinations thereof.

5. The stent coating of claim 1, wherein the biocompatible polymer is a biostable polymer selected from the group consisting of silicone, polyurethane, polyethylene, and polysulfone.

6. The stent coating of claim 5, wherein the biodegradable glass spheres are comprised of at least one alkali or alkaline earth metal oxide.

7. The stent coating of claim 6, wherein the alkali or alkaline earth metal oxide is selected from the group consisting of sodium oxide, potassium oxide, calcium oxide, magnesium oxide, and combinations thereof.

8. The stent coating of claim 7, wherein the therapeutic material is selected from the group consisting of anti-proliferative agents, anti-clotting agents, anti-plaque agents and combinations thereof.

9. A method of making a drug-eluting stent comprising:
providing a stent for implantation in a body lumen; and
applying to the stent a coating composition consisting essentially of biodegradable glass spheres containing a therapeutic agent and a biocompatible polymer.

10. The method of making the drug-eluting stent of claim 9, wherein the biodegradable glass spheres are comprised of at least one alkali or alkaline earth metal oxide.

11. The method of making the drug-eluting stent of claim 10, wherein the alkali or alkaline earth metal oxide is selected from the group consisting of sodium oxide, potassium oxide, calcium oxide, magnesium oxide, and combinations thereof.

12. The method of making the drug-eluting stent of claim 11, wherein the therapeutic agent is selected from the group consisting of anti-proliferative agents, anti-clotting agents, anti-plaque agents and combinations thereof.

13. The method of making the drug-eluting stent of claim 12, wherein the biocompatible polymer is a bioabsorbable polymer selected from the group consisting of poly(L-lactide), poly(D,L-lactide), polycaprolactone, polyoretheresters and nylon.

14. The method of making the drug-eluting stent of claim 12, wherein the biocompatible polymer is a biostable polymer selected from the group consisting of silicone, polyurethane, polyethylene, and polysulfone.
